(19) United States
(12) Patent Application Publication
(10) Pub. No.: US 2011/0288576 A1
(43) Pub. Date: Nov. 24, 2011

(54) Hoffy Bougie Gastric Tube

(76) Inventor: Jason Lane Hoffman, Weatherford, TX (US)

(21) Appl. No.: 12/785,115

(22) Filed: May 21, 2010

(51) Int. Cl. A61M 29/02

(52) U.S. Cl. 606/192

(57) ABSTRACT

A flexible bougie tube having a proximal end that has an opening for receiving a fluid and a distal end that is sealed for insertion into a patient having LSG a bariatric weight loss surgical procedure. The tube has a channel which extends from the opening in the proximal end to the distal end that is sealed, and at least one perforation or opening which communicates with the channel proximate the distal end of the flexible tube that is sealed to allow fluid received by the opening at the proximal end of the tube to pass thru the flexible tube. The bougie tube performs three functions of Ensuring patency of the esophagus and that the integrity of the entire stomach remains attached to the esophagus; Serving as a guide for the amount of the stomach that can be removed; and Testing the suture line integrity while remaining is place.

### BACKGROUND OF THE INVENTION

1. Field of the Invention

The present invention relates to a medical device which is used for insertion into a patient while under going a Laparoscopic Sleeve Gastrectomy (LSG) bariatric weight loss surgical procedure.

2. Description of Related Art

Obesity is a major health problem In the United States where complications of obesity affect well over two in five individuals. In practically every case obesity is directly related to overeating.

One method of reducing obesity includes a surgical procedure which reduces the volume of the stomach or limits the size of the stomach by means of bariatric surgery. There are three types of bariatric weight loss surgery procedures more specifically identified as the Laparoscopic Band (LB) procedure, the LSG procedure, and the Laparoscopic Roux En-Y Bypass (LRB) procedure.

The LB procedure reduces the amount of food that can be eaten at one time. A LB is wrapped around the upper part of the stomach, dividing the stomach into a small upper pouch that holds about one-half of a cup of food and a larger lower stomach. After the procedure the recipient feels full sooner and stays full longer. This procedure allows for the normal digestion and absorption of food.

The LSG procedure decreases the size of the stomach which limits the amount of food that can be eaten at any one time. During this procedure a thin vertical sleeve of the stomach is created by using a stapling device and the rest of the stomach is removed. The sleeve is about the size of a banana. As a result, the recipient of the procedure feels full sooner and stays full longer. This procedure allows for normal digestion and absorption of food.

The Bypass procedure restricts food intake and the amount of calories and nutrients that the body absorbs. In this procedure, the surgeon creates a small stomach pouch and attaches a section of the small intestine directly to the pouch. This allows food to bypass a portion of the small intestine. Having a smaller stomach pouch causes the person to feel full sooner and to eat less. Bypassing part of the small intestine means that the body absorbs fewer calories.

Although each of the three procedures can be effective in achieving the goals of weight loss set by a person, it is believed that the LSG procedure may have fewer complications post-op and does not interfere with the absorption of food as occurs with the bypass procedure.

It is believed that the LSG procedure will be the mainstay procedure for decades to come.

During the initial stages of the Lap Sleeve procedure a bougie tube (thick solid rubber tube), which provides two main functions once it is passed through the esophagus of the patient and into the stomach. Initially, the bougie tube serves as a guide for the surgeon while transection takes place. Secondly, the bougie ensures patency of the esophagus so that complete transection of the stomach does not occur. After the stomach has been transected and removed, a “leak” test is performed to determine the integrity of the new smaller stomach suture line. The test procedure requires that the bougie tube be removed and a second tube, a naso-gastric tube be inserted into the stomach.

The problem is that the current bougie tube can be used for only two functions and the introduction of the naso-gastric tube into the stomach for the test poses a risk of damaging the newly formed suture line the surgeon has just created.

This invention is directed to a single tube, herein after referred to as the Hoffy Bougie Gastric Tube (HGBT) which can be used to perform all of the functions which now require the use of two separate tubes during a Lap Sleeve surgical procedure.

### SUMMARY OF THE INVENTION

In an exemplary embodiment of the present invention, there is disclosed a flexible bougie tube having a proximal end that has an opening for receiving a fluid and a distal end that is sealed for insertion into a patient having a LSG bariatric weight loss surgical procedure. The tube has a channel which extends from the opening in the proximal end to the distal end that is sealed, and at least one perforation or opening which communicates with the channel proximate the distal end of the flexible tube that is sealed to allow fluid received by the opening at the proximal end of the tube to pass thru the flexible tube. The Hoffy bougie tube performs three functions of—Ensuring patency of the esophagus and that the integrity of the entire stomach remains attached to the esophagus; Serving as a guide for the amount of the stomach that can be removed by the surgeon; and Testing the suture line integrity while remaining is place.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a schematic view of a bougie tube here disclosed which is located in a patient at the start of a LSG procedure and is used without being removed to perform three functions of Ensuring patency of the esophagus and that the integrity of the entire stomach remains attached to the esophagus; Serving as a guide for the amount of the stomach that can be removed; and Testing the integrity of the new stomach suture line in accordance with the principles of the invention; and

FIG. 2 is a perspective view of the new improved tube here disclosed in accordance with the principles of the invention.

### DESCRIPTION OF THE PREFERRED EMBODIMENT

During a LSG procedure a surgeon will normally require that a bougie tube be passed through the esophagus and into the stomach. This prior art tube currently serves two functions. One function is to ensure patency of the esophagus and that the integrity of the entire stomach will remain attached to the esophagus. The other function is to serve as a guide to the surgeon for the amount of the stomach that can be removed. The prior art Bougie tube used during a LSG procedure is a solid siliconized rubber tube which is soft and flexible. Once the stomach has been transected, the prior art bougie tube is removed. The success of the operation depends on the integrity of the new smaller stomach suture line. If there is a “leak”, it must be detected before the operation is completed. Most surgeons will request a “test” be performed by the anesthesia provider.

The test consists of removing the bougie tube and introducing a gastric tube, often referred to as a naso-gastric tube (NGT) into the stomach. This can pose a problem if the anesthesia provider passes the NGT tube through the new stomach suture line because the surgeon will have to fix the puncture.

If that does not occur, the surgeon clamps the distal portion of the stomach and the anesthesia provider injects air through the NGT tube. The surgeon looks for air bubbles, much like checking a flat tire for a leak. Most surgeons will require a second test through the same tube which consists of the anesthetist injecting a “blue dye” into the stomach so that he can verify that there is no blue liquid seeping through the new stomach suture line. If both tests are negative, the procedure is completed and the operation is a success at that time.

An NGT tube is usually marked to ensure that the tube has been inserted far enough into the patient's stomach. Many commercially available stomach tubes have several standard depth markings such as, for example 18″, 22″, 26″ and 30″ from its distal end. The end of the tube which is usually made of plastic may be lubricated before being inserted. The tube should be directed aiming down and back as it is moved down into the throat. When the tube enters the oropharynx and glides down the posterior pharyngeal wall, the patient if awake, may gag. Once the tube is past the pharynx and enters the esophagus, it is easily inserted down into the stomach.

The problem is that the current bougie tube which is used during a LSG procedure can be used for only two of the required three functions and the introduction of the NGT tube imposes a risk of damaging the newly formed suture line the surgeon has created.

The bougie tube here disclosed can be used for all three functions which are needed during a LSG procedure.

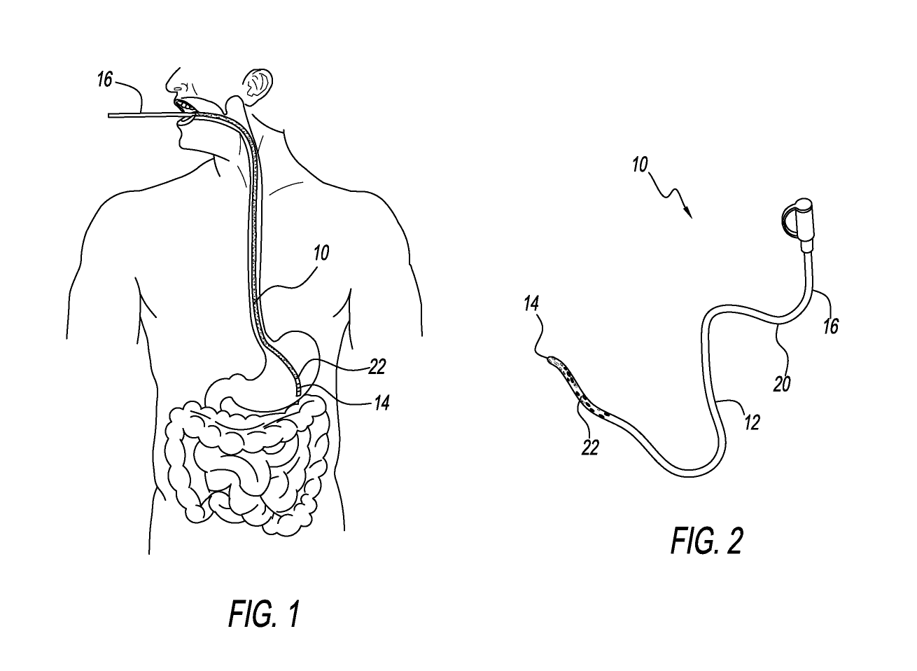

Referring to FIGS. 1 and 2, there is shown a view of the new improved bougie tube which can be used for all three functions during a LSG procedure and which eliminates the need to insert an NGT tube into a patient to confirm the integrity of the new stomach suture line during a bariatric surgery procedure.

The HBST tube 10 consists of an elongated tube portion 12 having a distal end 14 and a proximal end 16. The HBGT tube 10 is adapted to be installed in a patient so that it extends through the esophagus and into the stomach of a patient.

The tubular shaft 12 is an elongated tubular member having an outer wall 18 and a channel 20 which extends longitudinally through the tube 12. The tube 12 is made of silicon and rubber with sufficient flexibility to bend without applying excessive pressure on the tissues of a patient as it passes through to the patient's stomach.

Specifically, the elongated tube portion 12 can have a very flexible section which can expand as a result of the internal pressure of the testing fluid to occlude the esophagus during testing to reduce the likely chance of regurgitation of the test solution of fluid such as air or a liquid blue dye without significantly reducing the patients ability to breath.

Perforations or openings 22 are located in the wall of the elongated tube 12 on the distal end 14 which communicate with the channel 20. If desired the section of the elongated tube which includes the perforations or openings can be constructed with an enlarged cross sectional dimension relative to the dimension of channel 20. The cross-sectional area of the perforations or openings is preferably slightly less than the cross-sectional area of the channel 20 to slightly restrict the flow of a fluid such as air and/or a dye from channel 20 through the perforations or openings 22. This slight restriction will cause the pressure of the fluid in the channel to increase. The outer wall 18 is preferably constructed so that it is sufficiently flexible to permit the passage thereof to a patient stomach during the installation of the tube into the patient.

The bougie gastric tube disclosed serves as the surgical guide during a LSG procedure to performed three functions and can be either disposable or reusable. The initial two functions of the bougie gastric tube are; A) Ensuring patency of the esophagus and that the integrity of the entire stomach will remain attached to the esophagus; and B) To serve as a guide to the surgeon for the amount of the stomach that can be removed. The third function is the bougie gastric tube's ability to be used to test the suture line integrity while remaining in place thus eliminating the risk which is present with prior art tubes of introducing a separate tube into the patient that could puncture the new stomach suture line.

Additionally the new improved tube disclose eliminates the step in the “testing” process of requiring the bougie tube to be removed and a second tube be inserted into the patient with the result that the total time required for the surgical procedure is reduced and the patient is under sedation for a shorted period of time.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A bougie tube comprising:
a flexible tube having a proximal end that has an opening for receiving a fluid and a distal end that is sealed for insertion into a patient having a LSG bariatric weight loss surgical procedure;
a channel located in the flexible tube which extends from the opening in the proximal end of the flexible tube to the distal end that is sealed; and
at least one perforation or opening which communicates with the channel located in the flexible tube proximate the distal end of the flexible tube that is sealed to allow fluid received by the opening at the proximal end of the tube to pass thru the flexible tube.

2. The bougie tube of claim 1 wherein the fluid is air.

3. The bougie tube of claim 1 wherein the fluid is a liquid.

4. The bougie tube of claim 3 wherein the liquid is a dye.

5. The bougie tube of claim 3 wherein the dye is blue.

6. The bougie tube of claim 1 wherein the flexible tube has a diameter that occludes the esophagus only during testing when air or a liquid is moving thru the channel in the flexible tube to reduce the chance of regurgitation of the air of liquid.

7. A method of using a bougie tube during a surgical procedure comprises:
providing a flexible tube having a proximal end that has an opening for receiving a fluid and a distal end that is sealed for insertion into a patient having a LSG bariatric weight loss surgical procedure;
locating a channel in the flexible tube which extends from the opening in the proximal end of the flexible tube to the distal end that is sealed; and
providing at least one perforation or opening which communicates with the channel located in the flexible tube proximate the distal end of the flexible tube that is sealed to allow fluid received by the opening at the proximal end of the tube to pass thru the flexible tube.

8. The method of claim 7 wherein the fluid is air.

9. The method of claim 7 wherein the fluid is a liquid.

10. The method of claim 9 wherein the liquid is a dye.

11. The method of claim 9 wherein the dye is blue.

12. The method of claim 7 wherein the flexible tube has a diameter that occludes the esophagus only during testing when air or a liquid is moving thru the channel in the flexible tube to reduce the chance of regurgitation of the air of liquid.

13. The method of claim 7 wherein the bougie tube performs three functions of A) Ensuring patency of the esophagus and that the integrity of the entire stomach remains attached to the esophagus; B) Serving as a guide for the amount of the stomach that can be removed; and C) Testing the suture line integrity while remaining is place.
